(19) United States
(12) Patent Application Publication
(10) Pub. No.: US 2026/0207514 A1
(43) Pub. Date: Jul. 23, 2026

(54) Clofazimine Mini-Tablets for Treatment of Tuberculosis in Pediatrics

(71) Applicant: Via Therapeutics, LLC, Austin, TX (US)

(72) Inventors: Zachary WARNKEN, Austin, TX (US); Andrea TREMENTOZZI, Austin, TX (US); Patricia MARTINS, Austin, TX (US); John J. KOLENG, Austin, TX (US); Hugh SMYTH, Austin, TX (US); Ashlee BRUNAUGH, Ann Arbor, MI (US)

(21) Appl. No.: 19/138,736

(22) PCT Filed: Dec. 15, 2023
(86) PCT No.: PCT/US23/84455

§ 371 Date: Jun. 13, 2025

Related U.S. Application Data
(60) Provisional application No. 63/433,438, filed on Dec. 17, 2022.

(51) Int. Cl. A61K 9/20; A61K 31/498; A61K 47/14; A61K 47/20; A61K 47/38; A61P 31/06

(52) U.S. Cl. CPC A61K 9/2086; A61K 31/498; A61K 47/14; A61K 47/20; A61K 47/38; A61P 31/06

(57) ABSTRACT

Clofazimine (CFZ) is an important component of the World Health Organization's (WHO) recommended all-oral drug regimen for treatment of multi-drug resistant tuberculosis (MDR-TB). However, the lack of a dividable oral dosage form has limited the use of the drug in pediatric populations, who may require lowering of the dose to reduce the likelihood of adverse drug events. Pediatric-friendly CFZ mini-tablets prepared from micronized powder via direct compression at a relatively high drug load provide a suitable dosage form for children as young as 6 months of age and patients that have difficulty swallowing whole capsules or tablets.

### CROSS REFERENCE TO RELATED APPLICATIONS

This application is a U.S. national stage application from PCT Application PCT/US23/084455 filed Dec. 15, 2023, entitled “Clofazimine Mini-Tablets for Treatment of Tuberculosis in Pediatrics” which claims priority and benefit from U.S. Provisional Patent Application No. 63/433,438 filed on Dec. 17, 2022, entitled “Low-Cost, Shelf-Stable, Patient-Adaptable Clofazimine Mini-Tablets for Treatment of Tuberculosis in Pediatrics,” the contents of both of which are incorporated in their entirety herein by reference.

### TECHNICAL FIELD

Embodiments of the subject matter disclosed herein generally relate to dosage forms and formulations of the pharmaceutical agent clofazimine, and more particularly to mini-tablet formulations of clofazimine for treatment of tuberculosis in pediatrics.

### BACKGROUND OF THE INVENTION

Tuberculosis (TB) contributed to the deaths of over 200,000 children in 2018. Increasingly, Mycobacterium tuberculosis strains exhibit resistance to first-line antibiotics, which has necessitated the incorporation of older, second-line antibiotics into treatment regimens that frequently exhibited a more toxic adverse effect profile. As part of an effort to develop an all-oral drug regimen for multi-drug resistant (MDR-TB) and extensively drug resistant (XRD-TB) tuberculosis strains, the World Health Organization (WHO) is now recommending the inclusion of the off-patent drug clofazimine (CFZ) into regimens (WHO, 2019).

Clofazimine is an anti-infective medicine most commonly used to treat leprosy under the brand name Lamprene. Clofazimine has also been utilized clinically for the treatment of non-tuberculous mycobacterium (NTM) infections such as Mycobacterium avium complex (Field et al., 2003; Jarand et al., 2016) and Mycobacterium abscessus (Yang et al., 2017; Jarand et al., 2011) including in pediatric patients (Martiniano et al., 2017; Adler-Shohet et al., 2019). In the 2019 small cohort study published by Adler-Shohet et al., patients of an average age of 5.8 years were dosed with 1 mg/kg of CFZ per day for an average of 105.6 days for the treatment of odontogenic M. abscessus infection. Adverse effects were limited to skin discoloration and gastrointestinal symptoms; however, these did not lead to discontinuation of therapy. Furthermore, the incorporation of CFZ in the treatment regimen enabled a decrease in the dosage of amikacin, which is associated with severe toxicities.

Clofazimine was FDA approved for the oral route in 1986 as the product Lamprene®, however, this product has since been withdrawn from the market and there is currently no commercially available formulation for CFZ within the United States. The existing CFZ formulation available elsewhere is capsule based and consists of micronized drug suspended in a waxy lipid matrix. This formulation cannot be accurately divided to allow for the dose adjustment that is required for treatment of pediatric patients. An additional complication is the known tissue accumulation and slow clearance of CFZ from the body (Baik et al., 2012; Swanson et al., 2015; Brunaaugh et al., 2022) which makes it difficult to correct for any toxicity caused by inadvertent overdosing. In the previously described case studies treating M. abscessus infection, the 1 mg/kg/day dose was achieved only through extension of the dosing interval to 2-6 days/week depending upon patient weight. Such an approach is widespread in clinical practice and often results in an undesirably complicated regimen and a potential increase in dosing errors. Thus, a dose-adjustable oral formulation of CFZ would represent a significant advance in the treatment of pediatric TB and NTM infections.

The development of a pediatric-friendly CFZ dosage form for TB carries additional considerations. Tuberculosis is more common in countries with developing economies. In these areas, access to off-the-shelf sweeteners or clean drinking water may be limited which can make medication administration more challenging for caregivers. Furthermore, regions with a high incidence of pediatric TB may have distinct culture differences to North America or Western European settings, which influence taste and color preferences; thus, a more generalizable formulation platform may be preferred in drug product development in order to adapt to local preferences (Craig et al., 2009). Environmental factors and their effects on drug stability must also be considered, such as high humidity and high heat. While a number of oral delivery technologies have been identified as having good acceptability for pediatric patients and are now commercially available, including solutions, syrups, suspensions, chewable tablets, orally disintegrating tablets, or thin films (Strickley et al., 2008), such dosage forms typically require higher transportation costs or refrigerated storage (in the case of liquids) or extensive moisture protection (in the case of rapidly dissolving solids), which is cost-prohibitive and infeasible for the high-humidity, resource-poor environments that have a large percentage of pediatric TB cases. The dramatic red staining that occurs with prolonged use of CFZ also precludes the use of pediatric oral dosage forms that are in liquid state or require prolonged contact with the oral mucosa in solid state forms. The preferred alternative is the development of a shelf-stable solid dosage form that exhibits minimal release of CFZ for oral administration, while enabling sufficient dissolution within the gastric fluid to exhibit bioequivalence to the existing FDA-approved CFZ capsules.

The greatest barrier to commercialization of TB therapy is cost. There is a strong inverse association between TB incidence and per capita GDP (WHO, 2016) and access to trained healthcare professionals in these regions is limited. Therefore, for successful implementation of an oral CFZ product for the treatment of pediatric patients to occur, the production cost must be as low as possible, and administration of the product must be simple, intuitive, and robust to patient errors. Brunaugh and colleagues have previously utilized air jet milling, a micronization technique that reduces size distribution through particle-particle collisions without requiring organic solvents or moving equipment parts (Brunaugh et al., 2018), for the development of a cost-effective, inhaled clofazimine formulation that enhances drug targeting to infected macrophages and minimizes systemic exposure (Brunaugh et al.; 2022; Brunaugh et al., 2010). While this targeted therapeutic approach may be beneficial for adult patients, a substantial portion of pediatric patients develop disseminated TB infections, which is an increased risk in children younger than 3 years of age (Cruz, 2010).

While the aforementioned jet milled clofazimine formulation was developed for inhalation, Brunaugh demonstrated that the micronized particles suspended using only polysorbate 80 in a normal saline solution exhibited sufficient oral bioavailability to significantly reduce bacterial burden in the lungs and spleen once steady state was reached, without the need for the lipidic excipients that are present in the original Lamprene® formulation (Brunaugh, 2022). In this case, the larger surface area resulting from reduction in particle size distribution likely increases the rate of dissolution within the acidic stomach environment, enabling absorption to be achieved upon transit to the small intestine. Thus, these data demonstrate the feasibility of using particle size reduction, rather than more complex dissolution enhancing approaches like amorphous solid dispersions, to improve oral bioavailability of poorly water soluble CFZ.

Mini-tablets are a newer dosage form, and are generally defined as tablets exhibiting a diameter of approximately 1-5 mm. Their small size makes them suitable for administration to patients as young as 6-months of age (Klingmann, 2017) who would otherwise be unable to swallow a standard tablet, and the very small size of the tablets does not require the incorporation of moisture-labile oral dispersion component to the formulation (Preis, 2015). In a randomized cross-over design, 4-mm tablets were found to have increased acceptability and preference over other typically utilized dosage forms (powder, suspension, syrup) in infant and pediatric children (van Riet-Nales et al., 2013). Several mini-tablet or granule-like formulations have been marketed (Gerrard et al., 2019), which indicates that acceptability of this dosage form from a commercial and regulatory standpoint.

Until a patient is of a size and age to effectively take standard dosage forms, a mini-tablet dosage form of CFZ that enables treatment with weight-based dose adjustment in children as young as 6 months of age is needed that also avoids staining of the teeth and mouth with longer term use.

### SUMMARY OF EXAMPLE EMBODIMENTS

According to an embodiment, a mini-tablet of a pharmaceutical for the treatment of tuberculosis is disclosed that includes a quantity of micronized clofazimine with at least one disintegrating agent to promote disintegration; at least one lubricant to prevent capping and wherein the mini-tablet has a diameter of about 5 mm or less.

According to another embodiment, a clofazimine mini-tablet based method of treating diseases caused by Mycobacterium tuberculosis is disclosed where the method includes determining a therapeutically effective patient dosage of clofazimine based upon patient age, weight, and disease indication; determining the quantity of mini-tablets per patient dosage based upon the dose of clofazimine per mini-tablet, and wherein each mini-tablet includes a quantity of micronized clofazimine; at least one disintegrating agent to promote disintegration; and at least one lubricant to prevent capping. The mini-tablet has, in preferred embodiments, a diameter of about 5 mm or less and is administered to the patient as a plurality of mini-tablets.

According to another embodiment, a method of manufacturing clofazimine mini-tablets is disclosed which includes the steps determining a target therapeutic dosage quantity depending generally on patient weight, age and indication, which in preferred embodiments also includes about a 50% w/w of clofazimine (CFZ). The CFZ drug substance is micronized and in preferred embodiments, the CFZ material is micronized by air jetting milling, the micronized CFZ is then blended with excipients to impart the desired disintegration time and hardness in the final tablet which in preferred embodiments includes a combination of disintegration agents to reduce disintegration time to about 2 minutes or less. The disintegrants in preferred embodiments include a first disintegrant and a second disintegrant. The blend may include a surfactant such as sodium lauryl sulfate, and for tableting, a lubricant such as sodium stearyl fumarate. Approximately two (2) mm round miniature tablets are then directly compressed to a target hardness which may then be coated.

### BRIEF DESCRIPTION OF THE DRAWINGS

The accompanying drawings, which are incorporated in and constitute a part of the specification, illustrate one or more embodiments and, together with the description, explain these embodiments. In the drawings:

FIG. 1 shows a table of disintegration and capping performance data for clofazimine mini-tablets produced with various excipient blends.

FIG. 2 shows an exemplary dissolution profile for clofazimine mini-tablets.

FIG. 3A shows a schematic describing the process for combining active minitablets with placebos and then coating the clofazimine mini-tablets at varying coated weight gains.

FIG. 3B shows exemplary simulated saliva-based disintegration testing of coated mini-tablets.

FIG. 4 shows a release profile of CFZ from the optimized coated mini-tablets assessed in FeSSGF and FeSSIF.

FIGS. 5A-5E show single dose plasma concentration-time profiles of clofazimine (CFZ) administered via two different oral formulations in adult Sprague-Dawley routes.

FIG. 6 shows a summary overview of an exemplary method of manufacturing clofazimine (CFZ) mini-tablets.

### DETAILED DISCUSSION OF EXAMPLES OF THE INVENTION

The following description of the embodiments refers to the accompanying drawings. The same reference numbers in different drawings identify the same or similar elements. The following detailed description does not limit the invention. Instead, the scope of the invention is defined by the appended claims. The following embodiments are discussed, for simplicity, with regard to dosage forms and formulation of clofazimine and more particularly to mini-tablet dosage forms and formulations of clofazimine for therapeutic treatment of tuberculosis in pediatric patients. However, the embodiments discussed herein are not limited to such elements.

Reference throughout the specification to “one embodiment” or “an embodiment” means that a particular feature, structure, or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. The drawings are intended to be illustrative of the claimed features and results and unless stated otherwise are not to scale. Where a dimension of a given feature may be pertinent, the detailed description will indicate one or more examples of the range and units of said dimension where needed to enable the subject matter. Further, the described features, structures or characteristics may be combined in any suitable manner in one or more embodiments.

Table 1 describes the estimated number of mini-tablets that would be required in children up to a dose of 50 mg, which corresponds to the lowest soft gelatin capsule dosage available. This assumes a dose of 1 mg/kg/day (WHO, 2016) and minimum CFZ content of 2.5 mg (50% w/w for a 5 mg tablet core) per tablet. Twenty mini-tablets is generally regarded as the maximum number that is acceptable to take at a single time as reflected in the maximum number that can be dosed in a single instance using commercially available dose counters such as a Phillip-Medisize. As such, an approximate minimum dose of 2.5 mg of CFZ is needed per minitablet.

TABLE 1
Estimated dosing of CFZ mini-
tablets in children

| Age | Weight | Number of mini- |
|---|---|---|
| (years) | (kg) | tablets* |
| 2-3 | 12-14 | 5-6. |
| 4-5 | 16-18 | 6-7 |
| 6-8 | 20-26 | 8-10 |
| 8-10 | 26-32 | 10-13 |
| 10-14 | 32-50 | 13-20 |

In one example, a 2.5 mg CFZ drug load in mini-tablets that would be of a suitable size for a large range of ages; 2 mm, would require around a 50% w/w drug load of the micronized powder. The problem that needs to be overcome is that due to the hydrophobicity of poorly-water soluble drugs, the excipients required for various formulation strategies that must be incorporated typically results in lower drug loads between around 5-30% w/w. Thus, there is a need to overcome the drug loading limitations to enable a sufficient loading of micronized CFZ to treat a wide spectrum of pediatric ages.

Mini-tablet cores with 50% w/w micronized clofazimine of the present disclosure were developed and further coated to inhibit disintegration in neutral pH environments but also allow disintegration and dissolution of the drug to occur once the mini-tablets are transported to the stomach environment. Disintegration and dissolution assays of the mini-tablets were further coupled to a side-by-side diffusion cell to determine permeability of the released drug through porcine intestinal tissue. A pharmacokinetic study was performed in rats using both CFZ suspension and optimized CFZ mini-tablets to examine the effect of processing on CFZ bioavailability in vivo.

### Methods

Clofazimine degradation was assessed using the high pressure liquid chromatography (HPLC) method published in the United States Pharmacopeia monograph for the drug (U.S.P., 2021) The mobile phase consisted of a 65:35 mixture of acetonitrile and an aqueous buffer consisting of 4.5 g/L sodium dodecyl sulfate, 1.7 g/L tetrabutylammonium hydrogen sulfate and 21.8 g/L Na₂HPO₄ adjusted to pH 3.0. Flow rate was 1.0 mL/min and infection volume was 20 μL. A LiChromspher® RP-8 100 column (250×4.6, 5 μm) (Millipore Sigma) was used. Measurements were performed using an Agilent 1100 HPLC with diode array detector set at 280 nm. The method was evaluated for accuracy, precision, specificity, filter compatibility, and linearity, and demonstrated the capability of quantifying impurity peaks as low as 0.05% of the target analyte concentration and of accurately measuring the concentration of CFZ in the presence of tablet excipient blends. Hydrophobic PTFE 0.22 μm filters were used for sample filtration prior to analysis. The method was found to be linear from 70-130% of the target analyte concentrations (250 μg/mL) as well as from 0.05-2.00% (for organic impurities/related substances).

For quantitation of CFZ in plasma to establish pharmacokinetic parameters of the developed formulation, a liquid chromatography-mass spectroscopy (LC-MS/MS) was developed. Plasma samples (10 μL) were precipitated with 90 μL of 0.1% formic acid in acetonitrile and spiked with 10 μL of a 100 ng/ml diclofenac sodium solution prepared in methanol as an internal standard. Samples were vortexed for 1 minute followed by centrifugation at 16,900×g for 5 minutes at 4° C. The supernatant (50 μL) was diluted with 100 μL 80:20 methanol: acetonitrile. Samples were analyzed by liquid chromatography (Vanquish Flex UHPLC, ThermoScientific) with a tandem mass spectrometry detector (TSQ Altis, ThermoScientific) in positive selection reaction monitoring (SRM) mode for clofazimine precursor/product ions m/z 473/431. Separation was performed using a Hypersil Gold™ column (50 mm×2.1 mm, 3 micron, ThermoScientific) at 45° C. using a gradient method with mobile phases A and B consisting of 0.1% formic acid in water 0.1% formic acid in acetonitrile, respectively, elute at a flow rate of 0.5 mL/min. Mobile phase B was held at 40% for the first 0.2 min. followed by ramp to 95% over the next 0.8 min and held at 95% for 1 min. The percentage of mobile phase B was then decreased back to 40% over the following 0.1 min, and held for 2.9 min.

### Preparation of Micronized Clofazimine

Clofazimine (CFZ) (Midas Pharma GmbH) was micronized by Lonza Group AG using a 10-inch spiral jet mill at 3 kg/hr feed rate and 100 psi grinding pressure. Particle size distribution of the milled powder was determined using a laser diffractor (HELOS, Sympatec GmbH) with RODOS dry powder disperser set at 4 bar dispersion pressure and 20% rotation. In one example, the powder exhibited an X10 diameter of 0.66 μm, X50 diameter of 1.47 μm, and X90 diameter of 3.50 μm.

### Evaluation of Clofazimine and Excipient Compatibility

Stress testing of micronized CFZ in combination with commercially available tableting excipient blends was performed to establish potential mechanisms of drug degradation and streamline excipient selection for mini-tablet preparation. Two different blends were assessed: Prosolv® EasyTab SP (JRS Pharma) and Ludipress® (BASF). Magnesium stearate (1% w/w) was also added to samples containing Ludipress, as Ludipress does not contain a lubricant component, and lubrication may be necessary during the tablet compression process.

Additionally, Eudragit® E PO was assessed, as a potential candidate for functional coating of the mini-tablets to prevent CFZ release in the saliva and subsequent staining of the mouth. Binary mixtures of micronized CFZ and excipient blends and micronized CFZ and Eudragit E PO were prepared at 1:1 weight ratios. Blends of CFZ with Prosolv EasyTab SP, Ludipress, or Eudragit E PO were left open or in closed HPDE vials and stored for 18 days at 70° C./20% relative humidity (RH) or 70° C./75% RH, which was the thermal equivalent of 6 months of storage. As a control, micronized CFZ alone and each excipient blend alone were stored open at each condition.

HPLC analysis was performed on day 0 for CFZ alone and CFZ with each excipient, as well as on day 18 for the same samples at each temperature and humidity condition. Assay values (% recovery) for all samples was determined by comparing peak area to a reference standard and correcting for sample water content measured by Karl Fischer titration method, and related substances were reported by relative retention time (RRT) to the reference CFZ peak.

### Preparation of Clofazimine Mini-Tablets

Direct compression of mini-tablets containing CFZ (50% w/w) in combination with commercially available tablet excipients at various ratios was performed using an TDP-5 tablet press (LFA Machines Oxford LTD) using a 2 mm diameter standard round punch and die (Natoli). Excipients and excipient blends were selected to support the direct compression process by acting as binders, glidants, disintegrants, lubricants, and/or wetting agents. The evaluated excipients included Prosolv® 90 (microcrystalline cellulose, colloidal silicone dioxide), Prosolv® EasyTab SP (microcrystalline cellulose, colloidal silicone dioxide, sodium starch glycolate, sodium stearyl fumarate), Starch 1500® (partially pregelatinized maize starch), Pharmaburst® 500 (SPI Pharma) (fructose and starch) Aerosil® (Enonik) (fumed silica) Ac-Di-Sol® (DuPont) (croscarmellose sodium), sodium stearyl fumarate (JRS Pharma), and sodium lauryl sulfate (Fisher Scientific). Tablets were evaluated for friability using a Vankel friabilator following guidance from USP <1216> Friability and hardness using a Pharma Test PTB-M500.

A YC-310 Mini Spray coater (Pilotech) was used to apply a taste-mask coating onto the prepared CFZ mini-tablets and placebo mini-tablets (prepared from Prosolv EasyTab SP). Spray nozzle bore was 0.8 mm, atomization air pressure was 0.08 MPa, air blower frequency was 33 Hz, inlet temperature was 45° C., and spray rate was 15 RPM. Active mini-tablets (5 g) were combined with 45 g of placebo mini-tablets and Eudragit E PO ReadyMix (Evonik) was sprayed onto mini-tablets over varying periods of time (10, 15, 20, and 25-min), which corresponded to 13%, 15%, and 23% weight gain.

### Assessment of In Vitro Drug Release and Permeation

Disintegration and dissolution of CFZ mini-tablets was characterized in several simulated biological fluids. Simulated saliva fluid (pH 6.8) containing 8.0 mg/mL sodium chloride, 0.19 mg/mL potassium phosphate monobasic, 2.38 mg/mL sodium phosphate dibasic was prepared according to a published recipe (32) and was used to assess disintegration of CFZ mini-tablets coated with Eudragit E PO ReadyMix. Disintegration testing took place in 20 mL borosilicate glass scintillation vials with 10 mL of media. Fasted-state simulated gastric fluid (FaSSGF) was obtained from a commercial vendor (Biorelevant.com Ltd, London, UK) and fed state simulated gastric fluid (FeSSGF) was prepared as described by Jantratid et al. (Dissolution media simulating conditions in the proximal human gastrointestinal tract: an update. Pharm Res. 2008; 25(7):1663-76, which is incorporated by reference herein for all purposes.) The preparation utilized a combination of an acetate buffer and whole milk to simulate the gastric conditions after a standard breakfast following FDA guidance (CPC, 2022). A preliminary assessment of CFZ saturation solubility in each media indicated that solubility in FaSSGF was 0.092±0.004 μg/mL, which was near the limit of quantitation of the HPLC method; therefore, gastric condition dissolution of uncoated and coated mini-tablets were assessed only in FeSSGF, in which CFZ saturation solubility was 20.1±1.86 μg/ml. The opacity of the FeSSGF media prevented visual assessment of mini-tablet disintegration; therefore, the gastric stage of this test was performed in FaSSGF.

Mini-tablet dissolution was also assessed in fed state simulated intestinal fluid (FeSSIF) (Biorelevant, London, UK). For all dissolution tests involving coated tablets in FeSSIF, 5 mini-tablets were initially placed in 1 mL of FaSSGF in microcentrifuge tube and gently inverted for 2 min to remove the pH-dependent coating. The uncoated tablets were then added to 150 ml of 37° C. FeSSIF in small sample container dissolution vessels with paddles rotating at 75 rpm (Distek, Inc., North Brunswick, USA). Concentrations of clofazimine were measured using HPLC at 5, 10, 15-, 30-, 60- and 120-min. Dissolution experiments were performed in triplicate.

Permeation of clofazimine across excised porcine intestine (Animal Technologies, Tyler, USA) was evaluated for both the optimized mini-tablet formulation as well as a suspension of the micronized CFZ using a side-by-side diffusion cell apparatus (PermeGear, Hellertown, USA). Coated mini-tablets were first subjected to 2 min of gentle inversion in 0.5 mL 0.01 N HCl media to ensure dissolution of the pH-dependent, taste-masking coating. A single uncoated mini-tablet was then added to the donor compartment of the side-by-side diffusion cells separated from the receptor compartment with excised porcine intestine orientated with the apical side of the membrane facing the donor compartment. For experiments using the micronized clofazimine suspension (6 mg/mL CFZ in 0.9% sodium chloride, containing 0.2% w/w polysorbate 80), the suspension was added directly to the donor compartment. The donor compartments were then filled with FeSSIF to a total volume of 10 mL 1-Decanol was selected as the receptor fluid, based upon its previous utilization by Warnken et al. (In Vitro-In Vivo Correlations of Carbamazepine Nanodispersions for Application in Formulation Development. J Pharm Sci. 2018; 107(1), which is incoporated by refernece herein for all purposes.) for assessment of in vitro-in vivo correlation, and its ability to provide a suitable sink condition for the poorly water soluble CFZ. The experiment was performed in triplicate for 24 hours, after which point the receptor fluid was diluted and CFZ content quantified using HPLC.

### Assessment of Single-Dose Pharmacokinetics in Rats

Oral administration of a suspension of micronized CFZ (25 mg/kg) to mice with chronic Mycobacterium tuberculosis (Mtb) infection was previously demonstrated to produce statistically significant reductions in lung and spleen bacterial burden after two weeks of dosing. We therefore sought to assess the bioequivalence of CFZ suspension and CFZ mini-tablets in male and female Sprague-Dawley rats (average weight 246 g) at two different dosing levels (10.3 mg/kg and 20.6 mg/kg) as a surrogate for a drug efficacy study. Three males and three females were used for each treatment/dosing group. A 6.25 mg/mL micronized CFZ suspension was prepared by suspending CFZ particles in normal saline with 0.32 mg/mL polysorbate 80 and dispersing the particles using a rotor stator homogenizer. To achieve the 10.3 mg/kg dose, 1.65 mL/kg of the suspension was administered via oral gavage, while to achieve the 20.6 mg/kg dose, 2.20 mL/kg of the suspension was administered via oral gavage. The optimized mini-tablets (containing 3 mg of CFZ) were placed in the cap only of a size 9 gelatin capsule and administered orally. To achieve the 10.3 mg/kg dose, 1 mini-tablet was administered, while 2 mini-tablets were administered to achieve the 20.6 mg/kg dose. A post-dose 1 mL saline flush was administered following mini-tab dosing. Blood samples were collected via tail venipuncture 0.5-, 1-, 2-, 4-, 6-, 24-, 48-, 72-, and 96-hours post-dose into tubes containing an anti-coagulant (sodium citrate). Tubes were stored on wet ice until processed to plasma by centrifugation (3500 RPM at 5° C. for 10 minutes) within 30 minutes of collection. The plasma samples were then transferred to new tubes and stored at −70° C. until analysis.

Single-dose PK parameters for each formulation and dosing level were determined using noncompartmental analysis in Phoenix WinNonLin Version (Certara). Statistical analysis of the resulting PK data to assess for bioequivalence was performed using the software R and followed procedures prescribed by the FDA (Statistical Approaches to Establishing Bioequivalence, 2001). Cₘₐₓ and AUC₀₋ₗₐₛₜ values for each subject in each treatment group were log transformed using natural logarithms, and a Welch's (unequal variance) two-sample t-test was performed to test the hypothesis that the true difference in the Cₘₐₓ or AUC₀₋ₗₐₛₜ means of the test product (i.e., CFZ mini-tablets) and the reference product (i.e., CFZ suspension) were not equal to 0. The 90 percent confidence interval for the difference in the means of the log-transformed data was determined.

### Results

Pediatric-friendly, dose-adjustable clofazimine mini-tablets can be generated using a low-cost direct compression process. Stress testing of powder mixes of micronized CFZ and commercially-available excipient blends for tableting was performed as a preliminary screen for formulation suitability. Prosolv Easytab SP and Ludipress excipient blends were selected as they are mixtures of several tableting excipients intended for direct mixture with active pharmaceutical ingredients to product tablets, and thus provided a rapid mechanism to screen several excipients for compatibility. Compared to baseline assay and impurity peak values, little to no degradation was observed for the drug-excipient blends under the conditions tested. Also, the tested excipients were further evaluated for their impact on critical quality attributes in the drug product, such as powder flow to fill the tableting die, disintegration, and tablet breaking force. Table 2 provides a summary of clofazimine-excipient (1:1) compatibility after storage at accelerated conditions for 18 days exemplifying the stability of the drug at relatively high temperatures and humidities with the selected excipients.

TABLE 2

| | Storage Conditions | | Assay | Impurity (% Area) | | |
|---|---|---|---|---|---|---|
| | | Container | % | RRT | RRT | RRT |
| Excipients tested | Temperature/RH | Closure | Recovery | 0.82 | 0.88 | 0.94 |
| Micronized CFZ | Initial | n/a | 95.61 | 0.096 | 0.109 | — |
| alone | 70 C./20% RH | Open | 101.32 | 0.113 | 0.117 | — |
| | 70 C./75% RH | Open | 100.29 | 0.096 | 0.126 | <0.05 |
| Prosolv Easytab | Initial | n/a | 100.32 | 0.101 | 0.117 | — |
| SP | 70 C./20% RH | Closed | 101.24 | 0.099 | 0.104 | <0.05 |
| (microcrystalline | | Open | 100.71 | 0.096 | 0.103 | <0.05 |
| cellulose, colloidal | 70 C./75% RH | Closed | 104.13 | 0.103 | 0.105 | 0.082 |
| silicon dioxide, | | Open | 100.49 | 0.102 | 0.103 | 0.064 |
| sodium starch | | | | | | |
| glycolate, sodium | | | | | | |
| stearyl fumarate) | | | | | | |
| Ludipress | Initial | n/a | 97.08 | 0.102 | 0.117 | — |
| (lactose | 70 C./20% RH | Closed | 91.80 | 0.095 | 0.106 | <0.05 |
| monohydrate, | | Open | 99.22 | 0.093 | 0.103 | <0.05 |
| povidone K30, | 70 C./75% RH | Closed | 99.48 | 0.097 | 0.105 | 0.056 |
| crospovidone) with | | Open | 97.52 | 0.096 | 0.106 | <0.05 |
| magnesium | | | | | | |
| stearate | | | | | | |
| Eudragit EPO | Initial | n/a | 101.00 | 0.096 | 0.11 | — |
| | 70 C./20% RH | Closed | 104.58 | 0.102 | 0.112 | — |
| | | Open | 102.72 | 0.098 | 0.11 | — |
| | 70 C./75% RH | Closed | 101.33 | 0.098 | 0.111 | — |
| | | Open | 105.39 | 0.1 | 0.116 | — |

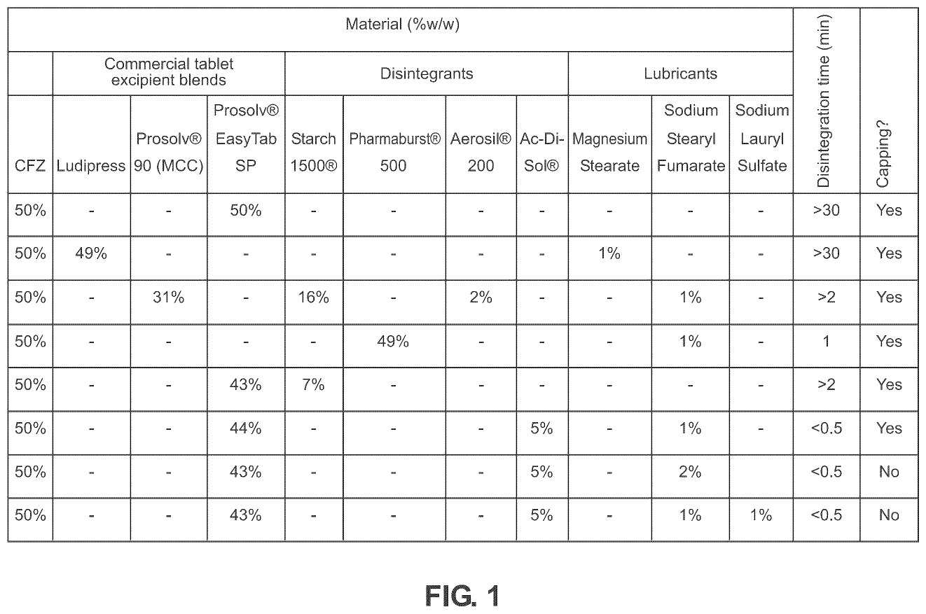

Embodiments of dosage forms and formulations of mini-tablets was targeted to enable a CFZ load of at least 50% w/w with rapid disintegration and dissolution in GI fluids to maximize oral bioavailability of this poorly water-soluble drug. Direct compression was initially performed using the specialty tablet excipient blends Prosolv® EasyTab SP or Ludipress® with magnesium stearate at a 50% w/w CFZ load; however, the resulting mini-tablets failed to disintegrate in FaSSGF and exhibited significant capping during production (see FIG. 1). The addition of excipients known to improve hardness and decrease capping without decreasing disintegration such as colloidal silicone dioxide and hydroxypropyl cellulose (data not shown) failed to solve the capping and disintegration issues of the generic tableting formulations.

Clofazimine mini-tablet compositions and their resulting tablet hardness and disintegration times are summarized in FIG. 1. Due to the high hydrophobicity of CFZ, drug loading of 50% w/w, and micron level of the drug particle size, it was unexpected to find that the incorporation of a combination of disintegrants into the formulation reduced the disintegration time to below 2 minutes while achieving a desired disintegration time at high drug loading. In one embodiment, the lowest disintegration time was achieved by adding 5% w/w croscarmellose sodium (Ac-Di-Sol®) to the blend containing sodium starch glycolate (see FIG. 1). Croscarmellose sodium was added to the blend containing sodium starch glycolate instead of increasing the amount of sodium starch glycolate to provide wicking action in addition to the swelling action of sodium starch glycolate. The combination thus provides an additive effect as compared to using just a single selected disintegration agent as the wicking action of the croscarmellose sodium can result in more rapid ingress of water into the dosage form where the sodium starch glycolate is present for swelling and disintegrating the tablet throughout.

Overcoming the minitablet disintegration time target through the use of the disintegrating agent combination was but one goal to produce a pharmaceutically acceptable dosage form; as tablet capping issues are also key. Tablet capping was overcome through the incorporation of additional lubrication, with either an increased percentage of sodium stearyl fumarate, the lubricant in the Prosolv EasyTab SP blend, or with a combination of sodium stearyl fumarate and sodium lauryl sulfate. Each of these formulations maintained the targeted rapid disintegration without incurring capping during the automated tableting process (see FIG. 1). It was not expected that the capping issue could be addressed while also achieving the target disintegration time given that adding addition components while maintaining 50% w/w micronized CFZ would not ordinarily be feasible.

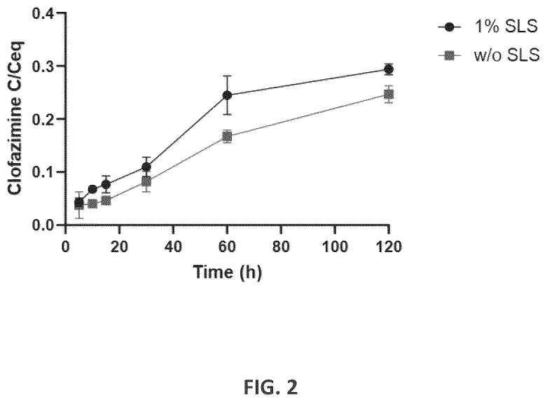

Embodiments of the aforementioned formulations containing either sodium stearyl fumarate alone or in combination with the anionic surfactant, sodium lauryl sulfate (SLS), were compared for dissolution rate improvements of CFZ in the mini-tablets in FeSSGF (pH 5.0). FIG. 2 shows dissolution profiles of CFZ mini-tablets containing 1% SLS and mini-tablets without SLS in FeSSGF media (pH 5.0, 37° C.). Error bars represent standard deviation, n=3. C/C_{eq}=the CFZ concentration at the time point normalized for the equilibrium solubility measured in the media.

Mini-tablets containing both sodium lauryl sulfate (SLS) and sodium stearyl fumarate (SSF) exhibited enhanced dissolution compared to those containing SSF alone (FIG. 2). Surfactants above their critical micelle concentration can solubilize drugs. In the event the SLS forms micelles which can solubilize CFZ during the dissolution test, the measured CFZ concentration in FeSSGF was normalized at each timepoint to the final saturation solubility of CFZ in the dissolution media to correct for any potential increases in saturation solubility of the drug from the SLS. The final average hardness of the mini-tablets containing the SLS was measured to be 10 N. The friability of the resulting mini-tablets was found to be 0.6%, deemed acceptable according to USP guidelines (USP <1216> Tablet Friability).

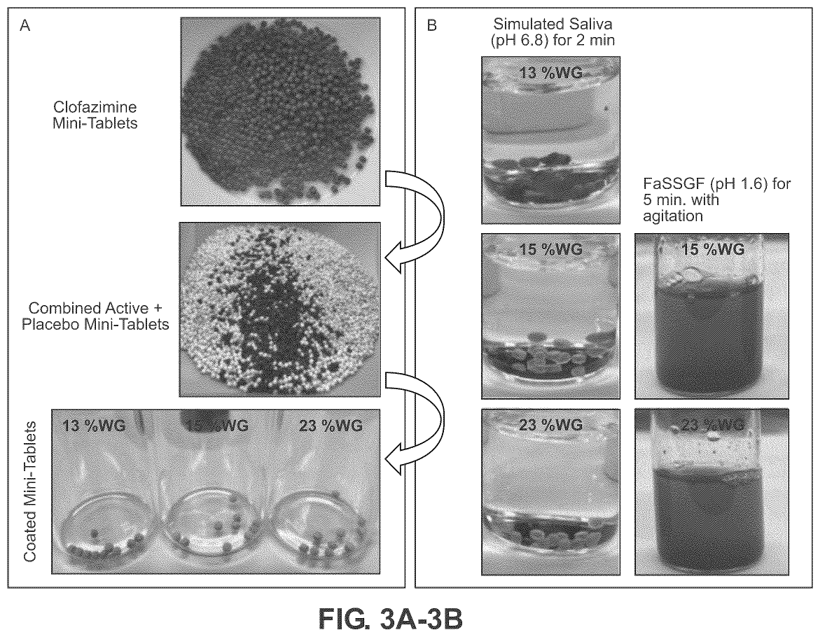

CFZ exhibits intense red pigmentation, which could result in staining of the mouth and teeth if the dosage form disintegrated and dissolved in the oral cavity prior to swallowing. Bitter taste of the drug may also reduce compliance in the pediatric population. Eudragit® EPO was therefore evaluated as a functional coating polymer to prevent premature mini-tablet disintegration and drug release. FIGS. 3A-3B provide a schematic describing the process for taking the active minitablets, combining them with placebos and then coating the clofazimine mini-tablets at varying coated weight gains (FIG. 3A). Disintegration testing of coated mini-tablets in first simulated saliva media (pH 6.8) followed by fasted-state simulated gastric fluid (FaSSGF) at pH 1.6 showing the inability of the 13% WG minitablets to sufficiently inhibit disintegration in the performed test. The effect of coating thickness on inhibition of mini-tablet disintegration in simulated saliva fluid (pH 6.8) was determined by subjecting mini-tablets to fluid bed coating for 10, 15 and 20 minutes to achieve weight gains of 13%, 15% and 23%, respectively (FIG. 3A). While the 13% weight gain coating level was insufficient to inhibit disintegration, the 15% and 23% weight gain levels inhibited disintegration in the simulated saliva fluid for at least 2 minutes. Transfer of the 15% and 23% weight gain mini-tablets to FaSSGF (pH 1.6) resulted in disintegration within 5 minutes and generation of a fine powder dispersion using a small amount of agitation (FIG. 3B).

### Release of Clofazimine from Optimized Mini-Tablets and Intestinal Permeation in Simulated Gastrointestinal Environments

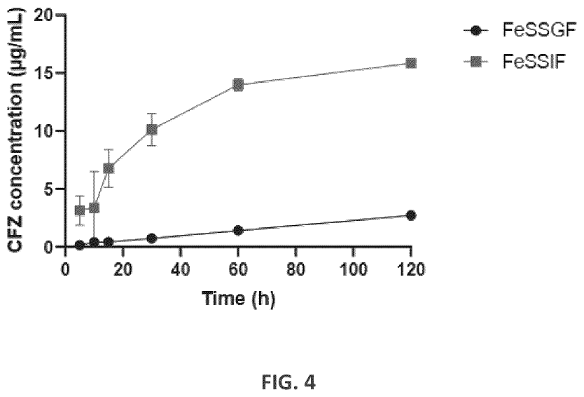

Release of CFZ from the optimized coated mini-tablets was assessed in both FeSSGF and FeSSIF. In the case of the fed-state intestinal dissolution testing, the mini-tablets were exposed to 0.1 N HCl for two minutes with gentle inversion to remove the coating before addition into the intestinal media. Mini-tablet dissolution in FeSSGF was slower than in FeSSIF, and the released CFZ remained below saturation solubility in this media (20.1±1.9 μg/mL). Alternatively, in FeSSIF, which contains bile salts available to assist in the wetting of the lipophilic micronized clofazimine, the released CFZ was able to reach saturation solution in the media (14.9±1.5 μg/mL) within 2 hours which provides the maximum opportunity for absorption without altering the physical form of the drug. FIG. 4 illustrates the resulting amount of clofazimine dissolved in the respective media up to two hours of testing. Dissolution profile of Eudragit EPO coated CFZ mini-tablets in both FeSSGF and FeSSIF media is thus shown. Error bars represent the standard deviation of n=3.

### Clofazimine Concentrations in Rats after Dosing with Minitablets Exceed Those Previously Reported to be Effective

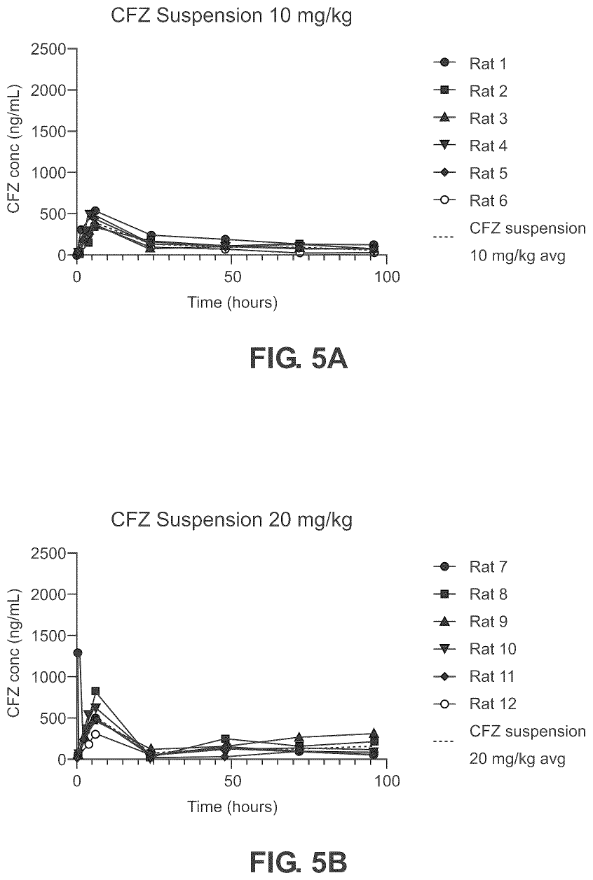

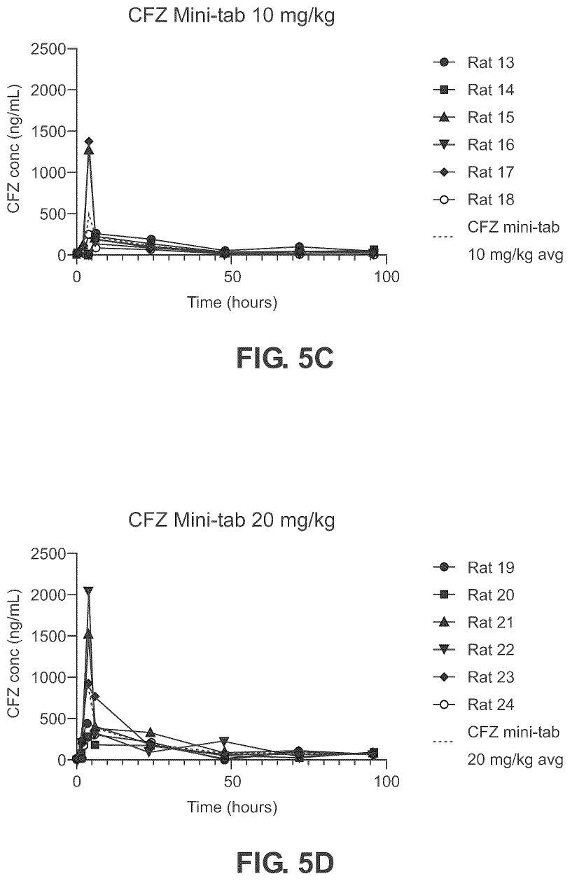

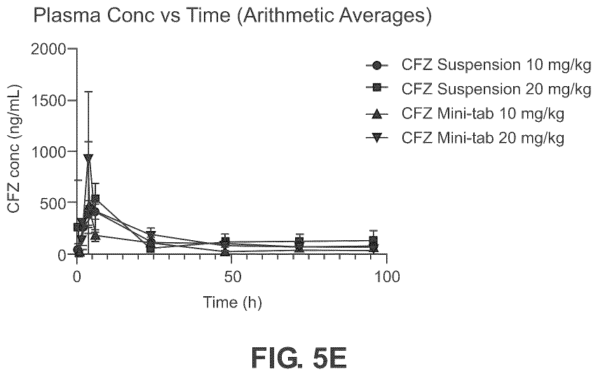

Single dose plasma concentration-time profiles of clofazimine (CFZ) administered via two different oral formulations in adult Sprague-Dawley routes are shown in FIGS. 5A-E. Serial blood samples were obtained after administration of a 10 mg/kg dose of CFZ suspension (FIG. 5A) or mini-tablets (FIG. 5B; single 2.5 mg mini-tablet) and a 20 mg/kg dose of CFZ suspension (FIG. 5C) or mini-tablet (FIG. 5D; two 2.5 mg mini-tablets), and the arithmetic mean CFZ plasma conc versus time profile for each treatment group (n=6) was determined. The arithmetic mean CFZ conc versus time for n=6 rats in each dosing group is presented in FIG. 5E. Pharmacokinetic results comparing 2.5 mg coated mini-tablets with a suspension of micronized clofazimine at 2 dose levels. The dashed red line in the zoomed in zero to 8 h time points represents the Cₘₐₓ reported in previous testing in BALB/c mice with the micronized suspension that was found to be effective in reducing Mycobacterium tuberculosis colony forming units (CFU). Error bars represent the standard deviation of n=6 Sprague-Dawley rats.

The results of the pharmacokinetic study shown in FIGS. 5A-5E are summarized in Table 3. The pharmacokinetic parameters were determined for each dosing group of either micronized clofazimine or coated mini-tablets in Sprague-Dawley rats.

TABLE 3

| | | Dose | T_{1/2} | Cₘₐₓ | Tₘₐₓ | AUC_{0-96 h} |
|---|---|---|---|---|---|---|
| Group | Test article | (mg/kg) | (h) | (ng/mL) | (ng/mL) | (ng*h/mL) |
| 1 | Micronized | 10 | 55.8 ± | 445.5 ± | 5.3 ± | 12975 ± |
| | Clofazimine | | 23.2 | 91.1 | 1.0 | 382.4 |
| | Suspension | | | | | |
| 2 | Micronized | 20 | 155.1 ± | 664.0 ± | 5.1 ± | 15559.3 ± |
| | Clofazimine | | 168.3 | 345.4 | 2.2 | 5381.3 |
| | Suspension | | | | | |
| 3 | Micronized | 10 | 42.3 ± | 588.6 ± | 5.0 ± | 6515.0 ± |
| | Clofazimine | | 21.3 | 564.3 | 1.1 | 3250.9 |
| | Coated | | | | | |
| | Mini-Tablet | | | | | |
| 4 | Micronized | 20 | 34.0 ± | 925.8 ± | 4.0 ± | 14393.7 ± |
| | Clofazimine | | 13.7 | 727.1 | 0.0 | 3944.7 |
| | Coated | | | | | |
| | Mini-Tablet | | | | | |

Generally, the clofazimine plasma concentrations were similar between the two formulations (suspension vs. mini-tablet). As can be seen in FIGS. 5A-5E, the dose is not directly proportional which is common for poorly water-soluble drugs and may be a result of the maximum amount of drug that can be dissolved in the gastrointestinal media has been exceeded. The overall exposure of the single mini-tablets in the rats was found to be lower than the other groups as a result of a few mice that quickly cleared the drug at early time points. The group dosed with 2 mini-tablets was found to result in a similar AUC_{0-96 h} as the equivalent dosed micronized suspension and with a higher but more variable Cₘₐₓ value. In all cases the Cₘₐₓ exceeded the reported Cₘₐₓ from a pharmacokinetic study in BALB/C mice that were treated at the dose found to be efficacious, providing promise that the mini-tablets can provide sufficient bioavailability for effective treatment of Mycobacterium tuberculosis infections. (Brunaugh, 2022).

### Methods of Manufacturing

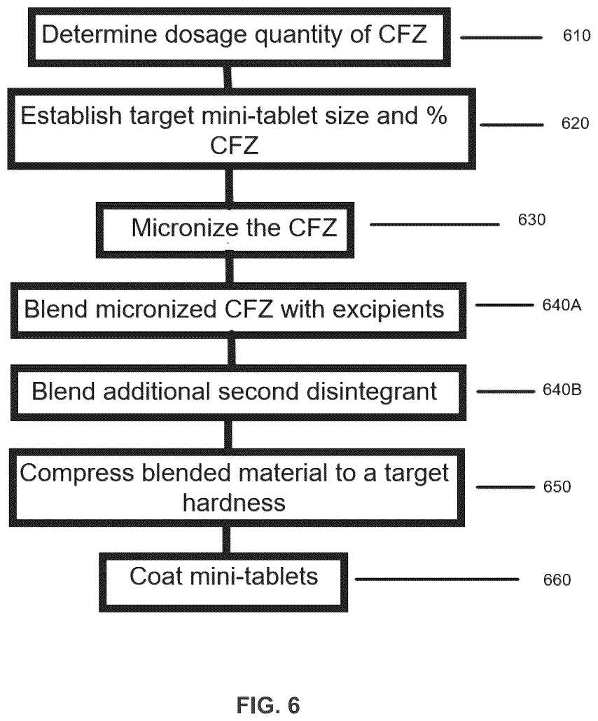

The following is a summary of an exemplary production method to produce the clofazimine minitablets as described herein and is outlined in FIG. 6. A therapeutic dosage quantity is first determined depending generally on patient weight, age and indication 610. A predetermined mini-tablet target size of about 2-5 mm is then established which in preferred embodiments also includes about a 50% w/w of clofazimine (CFZ) 620. The mini-tablet size target may also be a function of the target number of mini-tablets to be prescribed per dosage again depending on patient weight, age and indication. The CFZ drug substance is micronized 630, and in preferred embodiments, the CFZ material is micronized by air jetting milling.

The micronized CFZ is then blended 640A with excipients to impart the desired disintegration time and hardness in the final tablet which in preferred embodiments includes a combination of disintegration agents to reduce disintegration time to about 2 minutes or less. The disintegrants in preferred embodiments include a first disintegrant such as Prosolv EasyTab SP blend which contains sodium starch glycolate and a second disintegrant croscarmellose sodium (Ac-Di-Sol) which is blended until uniform with the CFZ. In certain embodiments the blend may include a surfactant such as sodium lauryl sulfate, and for tableting, a lubricant such as sodium stearyl fumarate. Note that the second additional disintegrant may be blended in an optional second blending stage 640B or combined with the first disintegrantinto a single blending stage 640A.

Approximately two (2) mm round miniature tablets are directly compressed to a target hardness 650. In certain embodiments, the target hardness is approximately 10 N. The mini-tablets are then coated 660 using a fluid bed coater such as Eudragit® EPO ReadyMix to a weight gain of approximately 23% in certain embodiments.

### Methods of Treatment

A clofazimine mini-tablet based method of treating diseases caused by Mycobacterium tuberculosis in a patient includes determining a therapeutically effective patient dosage of clofazimine based upon patient age, weight and disease indication; determining the quantity of mini-tablets per patient dosage based upon the dose of clofazimine per mini-tablet and administering to the patient the therapeutically effective amount of clofazimine as a plurality of mini-tablets. In this example, each mini-tablet comprises the agents discussed above which includes a quantity of micronized clofazimine; at least one disintegrating agent to promote disintegration; at least one lubricant to prevent capping; and a polymeric coating. In certain embodiments, each mini-tablet has a diameter of about 5 mm or less and in preferred embodiments, the diameter of 4 mm or less.

The clofazimine mini-tablet based treatment may be administered to patients suffering from chronic or acute Mycobacterium tuberculosis (Mtb) infections which may include multi-drug resistant (MDR-TB) and extensively drug resistant (XRD-TB) tuberculosis strains. Patients may include but not limited to pediatric TB sufferers as well as patients diagnosed with non-tuberculous mycobacterium (NTM) infections.

In one patient treatment example, a pediatric patient between 2 and 3 years of age may weigh 12.5 kilograms. Utilizing a weight-based dosing of 1 mg/kg/day of clofazimine as part of a treatment regimen the patient would require 12.5 mg clofazimine per day. Dosing five (5) 2.5 mg clofazimine mini-tablets once a day to the patient for the duration of their treatment would result in an accurate dosing for their weight.

Clofazimine has also been utilized clinically for the treatment of non-tuberculous mycobacterium (NTM) infections such as Mycobacterium avium complex and Mycobacterium abscessus including in pediatric patients. In pediatric patient populations suffering from these conditions, the mini-tablets of the present disclosure also address the lack of suitable dosage forms for these indications as well.

The foregoing embodiments are discussed, for simplicity, regarding oral dosage forms and formulations for pediatric tuberculosis treatment, however, the embodiments discussed herein are not limited to such elements or applications. Embodiments of the dosage forms and formulations disclosed herein have application to other treatment indications and patient populations. Further, the described features, structures, formulations, characteristics, and methods may be combined in any suitable manner in one or more embodiments.

What is claimed is:

1. A mini-tablet of a pharmaceutical for the treatment of tuberculosis comprising:
a quantity of micronized clofazimine;
at least one disintegrating agent to promote disintegration; and
at least one lubricant to prevent capping;
wherein the mini-tablet has a diameter of about 5 mm or less.

2. The mini-tablet of claim 1, wherein the quantity by weight of micronized clofazimine is about 50% or more of the total mini-tablet weight.

3. The mini-tablet of claim 1, wherein the micronized clofazimine has a mean particle diameter (X50) of 5 microns (μm) or less.

4. The mini-tablet of claim 1, wherein the micronized clofazimine has a mean particle diameter (X50) of 2 microns (μm) or less.

5. The mini-tablet of claim 1, wherein the number of disintegrating agents is two comprising a first agent and different second agent.

6. The mini-tablet of claim 5, wherein one disintegrating agent is an agent to promote swelling and the second agent is an agent to promote wicking.

7. The mini-tablet of claim 6, wherein the first disintegrating agent includes sodium starch glycolate and the second agent includes croscarmellose sodium.

8. The mini-tablet of claim 1, wherein the lubricant is a monoester of fumaric acid.

9. The mini-tablet of claim 1, wherein the lubricant further includes an anionic surfactant.

10. The mini-tablet of claim 1, further wherein the lubricant is a combination of both a monoester of fumaric acid and an anionic surfactant.

11. The mini-tablet of claim 10, further wherein the lubricant is a combination of both a monoester of fumaric acid and an anionic surfactant and further wherein the monoester of fumaric acid is sodium stearyl fumarate and the anionic surfactant is sodium lauryl sulfate.

12. The mini-tablet of claim 1, wherein the mini-tablet has a diameter of about 4 mm or less.

13. The mini-tablet of claim 1, further comprising a polymeric outer coating.

14. A clofazimine mini-tablet based method of treating diseases caused by Mycobacterium tuberculosis in a patient in need of treatment thereof, the method comprising:
determining a therapeutically effective patient dosage of clofazimine based upon patient age, weight, and disease indication;
determining the quantity of mini-tablets per patient dosage based upon the dose of clofazimine per mini-tablet,
wherein each mini-tablet includes a quantity of micronized clofazimine; at least one disintegrating agent to promote disintegration; at least one lubricant to prevent capping; and further wherein the mini-tablet has a diameter of about 5 mm or less; and
administering to the patient the therapeutically effective amount of clofazimine as a plurality of mini-tablets.

15. The clofazimine mini-tablet based method of claim 14, wherein the quantity by weight of micronized clofazimine of each mini-tablet is about 50% or more of the total weight of each mini-tablet.

16. The clofazimine mini-tablet based method of claim 14, wherein the micronized clofazimine of each mini-tablet has a mean particle diameter (X50) of 5 microns (μm) or less.

17. The clofazimine mini-tablet based method of claim 14, wherein the micronized clofazimine of each mini-tablet has a mean particle diameter (X50) of 2 microns (μm) or less.

18. The clofazimine mini-tablet based method of claim 14, wherein the number of disintegrating agents of each mini-tablet is two comprising a first agent and different second agent.

19. The clofazimine mini-tablet based method of claim 18, wherein one disintegrating agent is an agent to promote swelling and the second agent is an agent to promote wicking.

20. The clofazimine mini-tablet based method of claim 14, wherein the diseases caused by Mycobacterium tuberculosis are multi-drug resistant (MDR-TB) and extensively drug resistant (XRD-TB) tuberculosis.
